(19) United States
(12) Patent Application Publication
(10) Pub. No.: US 2003/0035869 A1
(43) Pub. Date: Feb. 20, 2003

(54) Identification method for purely natural products

(76) Inventors: Xiaowei Li, Hangzhou city (CN); Zuxun Yu, Hangzhou city (CN); Zheng Yang, Hangzhou city (CN); Jing Liang, Hangzhou city (CN)

Correspondence Address: RABIN & CHAMPAGNE, P.C., Suite 500, 1101 14 Street, N.W., Washington, DC, 20005, US

(21) Appl. No.: 10/216,921

(22) Filed: Aug. 13, 2002

(30) Foreign Application Priority Data
Aug. 18, 2001 (CN) 01126504.3

(51) Int. Cl. G01N 33/02

(52) U.S. Cl. 426/231

(57) ABSTRACT

An identification method used for distinguishing the sham from the genuine in a purely natural products, comprising to treat the specimen into a testable piece and count the number of rays released by each unit weight and unit time period by liquid flashing arithmometer; the disintegration value of C¹^{⁴} can be calculated through testing efficiency calibration, marked by dpm/gc. This dpm/gc can tell the percentage of C¹⁴ contained in the test piece. Compare the dpm/gc value of an approved natural specimen A with that of an unknown test piece B. If A is equal to or close to B, then the test piece is made of purely natural material(s); if A is far greater than B, then B is not made of 100% natural material. This method can be used in testing the purity of natural contents of Chinese medicine, western medicine, foodstuffs, food additives, health-protection food and flavorings.

The present invention relates to an identification method for purely natural products, such as traditional Chinese medicine, western medicine, foodstuffs, food additives, health-protection food and flavorings.

### BACKGROUND OF THE INVENTIONS

Most of the natural products vendors all declare that the products they sell are genuine pure natural products, and the raw material of their merchandise is directly collected or extracted from fresh plants or animals to prove that the product is purely natural. However this has been hard for the customer to distinguish via accessible methods. The current identification method in common use is based on comparing patterns of tested substances obtained by aerial chromatography or liquid chromatography with a standard chromatogram of an assured natural piece. Unfortunately the result of this comparison can only be used as a not very reliable reference, rather than a major deciding point of whether the tested material is collected from living life-forms.

### SUMMARY OF THE INVENTION

The present invention aims to provide an identification method for purely natural products, which can easily and precisely identify the purely natural products or not.

The present invention also aims to provide a method, which can further determine the percentage of the component of the synthetic composition in the products.

Genuine natural products are made or extracted from natural materials; while synthetic products are made through synthesis. The carbon ingredients of a real natural product comes from modern carbon, while that of a synthetic product comes from “dead” carbons in materials like coal, petrol and natural gas, which have been buried underground for tens of thousands of years. As known to all, among the isotopes of carbon, only C¹⁴ is radioactive, from which \hat{a} ray radiates out from C¹⁴ in its disintegration process to C¹³. The half-life of disintegration is 5,568 years. This method has been used in identifications for archaeological and geological purposes, by determining the radioactive nature of C¹⁴ by means of a liquid flashing arithmometer.

The manner of using the radiation of C¹⁴ identify the purely natural products is as follows:

STEP ONE: Treat the specimen into a testable piece and count the number of a rays released by each unit weight and unit time period by liquid flashing arithmometer, mark the value with cpm/g, i.e., number of a rays per minute/gram; the disintegration value of C¹⁴ can then be calculated through testing efficiency calibration, marked by dpm/gc. This dpm/gc can tell the percentage of C¹⁴ contained in the test piece.

STEP TWO: Compare the dpm/gc value of an approved natural specimen A with that of an unknown test piece B. If A is equal to or close to B, then the test piece is composed of purely natural material(s); if A is far greater than B, then B is not made of 100% natural material: the percentage of natural content can thereafter be calculated as B/A×100%.

This identification method is highly reliable, as the dpm/gc value of a pure natural specimen is greater by two digits (hundred times over) than specimen that contains non natural material.

### DETAILED DESCRIPTION OF THE PREFERRED EMBODIMENT

Embodiment One

Using the mensuration of C¹⁴ to identify whether a known specimen-“Natural” health-protection food Soybean is made of purely natural materials.

Test the disintegration value of a proved specimen of pure natural Soybean Isofavone to be 13.094 dpm/gc; then test the disintegration value of the known specimen of non natural contents to be 0.11 dpm/gc. The dpm/gc value of the proven specimen is much higher than that of the non-natural test piece. It can be proved that the non-natural piece consists of “dead” carbons that are 37,000 years old, and thus not natural.

Embodiment Two

Using the mensuration of C¹⁴ to test another specimen of Soybean Isofavone which is knowto contain 40% sythetic Soybean Isofavone and 60% purely natural Soybean Isofavone.

The dpm/gc value of a proved specimen of pure natural Soybean Isofavone is know from embodiment one to be 13.094 dpm/gc; then test the dpm/gc value of the impure specimen to be 7.921 dgm/gc. It can be proved that the percentage of the purely natural composition is

$$\frac{7.921}{13.094} \times 100 \% = 60 \% ,$$

while the percentage of the synthetic composition is 40%.

Embodiment Three:

Using the mensuration of C¹⁴ to identify whether a known specimen-“Natural” Ubiquinone-10food Soybean is made of purely natural materials.

Test the dpm/gc value of a proved specimen of pure natural Ubiquinone-10 extracted from animal hearts to be 13.085 dpm/gc; then test the dpm/gc value of the other specimen to be 13.085dgm/gc. The dpm/gc values of the two specimens are the same,so the latter specimen is purely natural.

Embodiment Four

Using the mensuration of C¹⁴ to identify whether a known specimen-“Natural” Ubiquinone-10 is made of purely natural materials.

Test the dpm/gc value of a proved specimen of pure natural Ubiquinone-10 extracted from animal hearts to be 13.085 dpm/gc; then test the dpm/gc value of the other specimen to be 0.23dgm/gc. The dpm/gc values of the two specimens are the different, so the latter specimen is not purely natural.

Embodiment Five

Using the mensuration of C¹⁴ to identify whether a known specimen-“Natural” “Natural” medicine Vitamin E is made of purely natural materials.

Test the dpm/gc value of a proved specimen of pure natural Vitamin E to be 13.807 dpm/gc; then test the dpm/gc value of the other specimen to be 13.807dgm/gc. The dpm/gc values of the two specimens are the same, so the latter specimen is purely natural.

Embodiment Six

Using the measuration of C¹⁴ to identify whether a known specimen-“Natural” medicine Vitamin E is made of purely natural materials.

Test the dpm/gc value of a proved specimen of pure natural Vitamin E to be 13.807 dpm/gc; then test the dpm/gc value of the other specimen to be 0.480dgm/gc. The dpm/gc values of the two specimens are different, so the latter specimen is not purely natural.

Embodiment Seven

Using the mensuration of C¹⁴ to identify whether a known specimen-“Natural” Sodium acetate is made of purely natural materials.

Test the dpm/gc value of a proved specimen of pure natural Sodium acetate to be 15.148 dpm/gc; then test the dpm/gc value of the other specimen to be 15.148dgm/gc. The dpm/gc values of the two specimens are the same, so the latter specimen is purely natural.

Embodiment Eight

Using the mensuration of C¹⁴ to identify wether a known specimen-“Natural” Sodium acetate is made of purely natural materials.

Test the dpm/gc value of a proved specimen of pure natural Sodium acetate to be 15.148 dpm/gc; then test the dpm/gc value of the other specimen to be 0.374dgm/gc. The dpm/gc values of the two specimens are different, so the latter specimen is not purely natural.

We claim

1. An identification method for purely natural products, characterized in that it comprises the steps of first to treat the specimen into a testable piece and count the number of \hat{a} rays released by each unit weight and in unit time period by means of a liquid flashing arithmometer, mark the value with cpm/g, which stand on number of a rays per minute/gram; the disintegration value of C¹⁴ can then be calculated through testing efficiency calibration, marked by dpm/gc, this dpm/gc can tell the percentage of Carbon 14 contained in the test piece, then compare the dpm/gc value of an approved natural specimen A with that of an unknown test piece B, if A is equal to or close to B, then the test piece is made of purely natural material; if A is far greater than B, then B is not made of 100% natural material.

2. The identification method according to claim1, characterized in that after discovering that the latter one is not genuine natural products, it can further determine that the component of the synthetic composition in the product is 100%-B÷A×100%.
